(19) United States
(12) Patent Application Publication
(10) Pub. No.: US 2024/0280455 A1
(43) Pub. Date: Aug. 22, 2024

(54) CURING KINETICS OPTIMIZATION MODULE (C-KOM) FOR AXIAL DYNAMIC MECHANICAL ANALYSIS

(71) Applicant: Soreq Nuclear Research Center, Yavne (IL)

(72) Inventors: Ronen Verker, Yavne (IL); Eliana Ruth Wallach, Yavne (IL); Yuval Vidavsky, Yavne (IL); Asaf Bolker, Yavne (IL); Irina Gouzman, Yavne (IL)

(73) Assignee: Soreq Nuclear Research Center, Yavne (IL)

(21) Appl. No.: 18/681,151

(22) PCT Filed: Aug. 10, 2022
(86) PCT No.: PCT/IB2022/057470

§ 371 Date: Feb. 5, 2024

Related U.S. Application Data
(60) Provisional application No. 63/232,302, filed on Aug. 12, 2021.

(51) Int. Cl. G01N 11/10

(52) U.S. Cl. CPC G01N 11/10

(57) ABSTRACT

A system for dynamic mechanical analysis of a polymer includes a stationary crucible for receiving therein a polymer, a movable plunger operative to apply shear forces on the polymer, and a dynamic mechanical analyzer (DMA) including a movable shaft coupled to the plunger.

### FIELD OF THE INVENTION

The present invention generally relates to a novel fixture setup which provides a fast path toward characterization and optimization of the curing and thermal degradation processes of polymers, such as thermosets, while using an axial dynamic mechanical analyzer.

### BACKGROUND OF THE INVENTION

Thermo-analytical study of thermoset resins, either during research and development or during quality assurance activities, involves the usage of diverse analytical equipment for characterization from initial mixing to final decomposition. Gelation and vitrification are usually measured using rheometers. Curing, post-curing, and curing kinetics are often studied using differential scanning calorimetry (DSC). Glass transition temperature (T_{g}) is measured via DSC or dynamic mechanical analysis (DMA) and finally thermal decomposition measurements are done using thermal gravimetric analysis (TGA). (Dynamic mechanical analysis (DMA) is a technique in which the elastic and viscous response of a sample under oscillating load, are monitored against temperature, time or frequency.)

### SUMMARY OF THE INVENTION

The present invention presents a new setup, denoted C-KOM, as well as a protocol for its usage, which unite diverse thermo-analytical techniques into one tool. As a case study, the inventors investigate via C-KOM the effect of various iso-thermal curing temperatures on the physical properties of an epoxy adhesive. Through C-KOM the inventors identify the effect of the curing temperature on the adhesives' gelation and vitrification points, as well as on its T_{g}. The data collected via C-KOM was used to extract the adhesive's curing reaction rates as well as activation energies and to compare and evaluate previously suggested curing procedures and assess their validity. As a final step the thermal decomposition temperature of the epoxy adhesive in hand was identified via C-KOM.

The novel C-KOM setup provides a fast path toward characterization and optimization of the curing processes of thermoset materials in a way that was not available before.

Thermosetting polymers (thermosets) are polymers that are permanently hardened by curing from a liquid or soft solid that contain monomers or oligomers. Due to the three-dimensional network of covalent bonds (crosslinking), thermosets are generally stronger and better suited to high temperature applications than thermoplastic materials.

Since thermosets are often used in high performance and safety critical applications and cannot be processed after hardening, every step of the lifecycle of thermosets is optimized and highly investigated. The lifecycle of thermosetting polymers contains three main steps: curing, operation (mainly dictated by the thermomechanical properties of the material), and degradation that results in the end of use of the material. Since each of these steps affects the processing and use of these materials, various testing methods are performed. Usually, each step is characterized and optimized separately by different analytical methods.

The curing process has a significant influence on final thermomechanical properties of the cured polymer. Since normal thermosets cannot be processed after curing, understanding of the cure mechanism is highly important for process optimization and is thoroughly investigated.

Different techniques are being used for the characterization and optimization of the curing and post-curing processes. Rheological evaluation of the resin, such as gelation and vitrification points, is of great interest, defining the pot life and the time in which the resin can be processed. The methods employed to monitor the progress of the curing process usually fall into two main categories: methods that monitor the changes in chemical functionality and methods that monitor the changes in the physical properties due to the formation of polymer chains and three dimensional crosslinked network. Monitoring the chemical functionality changes is usually done by spectroscopy methods. Since it is assumed that the rate of heat generation is proportional to the rate of the cure reaction, one of the methods mostly used in the prior art to study the kinetics of curing reactions is the thermal analysis by differential scanning calorimeter (DSC). In addition to the kinetics of the physiochemical processes, thermal analysis is also useful technique for making time-temperature correlation, and to optimize the variables of the curing process.

The curing process of a thermoset crosslinked network involves the transformation of low molecular species liquid to polymer network solid. Thus, gelation and vitrification are critical phenomena that govern the handling and processing of thermosets. The identification of the gelation point is important because beyond the gel point, the material is unable to flow and the processability of the material is greatly reduced. Consequently, injection molding, resin transfer molding (RTM), 3D printing and other enabling technologies are highly sensitive to the gelation point. Usually the gel and vitrification points are characterized by the changes in the rheological properties of the resin.

Following curing, the operation stage is mainly governed by the thermomechanical properties of the thermosets. Since above T_{g} thermosets lose abruptly their mechanical properties, it is highly important to avoid temperatures that approach the T_{g} of the polymers. In order to evaluate this parameter, in most cases T_{g} is measured using either DSC or dynamic mechanical analysis (DMA). Typically, DMA is more sensitive than DSC in characterizing the T_{g} and also provide valuable data regarding the mechanical properties of the material.

The degradation or failure of thermosets is dictated by the operational-induced stage and environmental conditions. The degradation process is the reduction in the thermo-physical properties caused by destructive changes in the chemical composition. Because thermosets are often being used in high performance applications, the ability to characterize and model their degradation and predict damage and failure in advance, is highly desirable. The most common analytical method used to study the thermal degradation of polymers is thermogravimetric analysis (TGA). TGA allows characterizing the degradation of polymers by measuring the mass loss of a sample at elevated temperatures. However, it does not produce in-situ data about the actual mechanical properties deterioration during this process.

Currently the full characterization of the whole lifecycle of thermosets requires several analytical techniques which are based on different experimental methods with various sample preparation procedures. Hence, the development of an analytical method that allows characterizing the whole lifecycle of thermoset materials in a single procedure is highly desirable. Such a method would facilitate the research of thermosets and accelerate the development process of novel materials.

The inventors present a novel axial DMA setup, the curing kinetics optimization module (C-KOM), a fixture which allows the investigation of the whole life cycle of a thermoset material from fluidic pre-cured resin, through operational solid, and finally its degradation. Axial DMAs are mainly designed for measuring solid and semisolid samples. They can be adapted to measure liquid specimens however; the inherent instrument design makes this inadvisable and may cause operational failures. C-KOM allows the usage of DMA without the concern of damaging the machine while characterizing the entire lifecycle of thermosets, from the curing and post curing processes to the evaluation of the thermomechanical properties and finally the thermal degradation of the materials. The use of C-KOM will allow faster research, development, and optimization of the processing and handling conditions of novel thermoset materials.

There is provided in accordance with an embodiment of the invention a system for dynamic mechanical analysis of a polymer, the system including a stationary crucible for receiving therein a polymer, a movable plunger operative to apply shear forces on the polymer, and a dynamic mechanical analyzer (DMA) including a movable shaft coupled to the plunger.

An elastic disc may be placed at a bottom of the crucible, wherein the plunger is preloaded against the elastic disc and configured to apply oscillatory normal forces against the elastic disc and shear forces against the polymer.

The plunger may be movable in oscillatory axial motion. The plunger may apply the shear forces on the polymer while the polymer is in a liquid state, a solid state, or a transition between a liquid state and a solid state.

There is provided in accordance with an embodiment of the invention a method for dynamic mechanical analysis of a polymer, the method including placing a polymer in a stationary crucible, applying shear forces on the polymer with a movable plunger, and performing dynamic mechanical analysis of the polymer with a dynamic mechanical analyzer including a movable shaft coupled to the plunger.

The method may further include exposing the crucible to variable temperatures or irradiating the crucible with electromagnetic radiation.

### BRIEF DESCRIPTION OF THE DRAWINGS

The present invention will be understood and appreciated more fully from the following detailed description taken in conjunction with the drawings in which:

FIGS. 1A and 1B. Schematics of the C-KOM apparatus (1A) and of C-KOM integrated with the DMA (1B).

FIG. 2. Molecular structure of the epoxy monomers; Epon 826 resin and Jeffamine D230 polyetheramine crosslinker.

FIGS. 3A and 3B. Shear force measurements during the multi-step curing process of the epoxy resin (3A) and zoom in on the first stage of the curing process (3B).

FIGS. 4A-4F. In situ curing monitoring via τ (FIGS. a-c) and dτ/dt (FIGS. d-f) vs. t at various isothermal curing temperatures.

FIGS. 5A, 5B and 5C. Gel time (5a) and vitrification time (5b) vs. isothermal curing temperature. (5c) Partial Time-temperature-transformation (TTT) isothermal cure diagram.

FIG. 6. Extraction of T_{g} from τ vs. temperature scans for different curing temperatures (a) and the attained T_{g} (onset) results vs. the isothermal curing temperature (b).

FIGS. 7A, 7B and 7C. Curing rate vs. degree of cure. Comparison between experimental data and model prediction (a). Arrhenius plots of reaction order (b) and reaction rate constants (c) obtained via C-KOM for isothermal curing.

FIG. 8. Arrhenius type plot to obtain Eₐ of the crosslinking reaction, based on t_{gel} at different isothermal curing temperatures.

FIG. 9. Comparative thermo-analysis, between DSC and C-KOM, during curing and post-curing of the epoxy adhesive.

FIG. 10. Comparative thermal decomposition, between TGA and C-KOM, during heating of the epoxy adhesive.

### DETAILED DESCRIPTION OF EMBODIMENTS

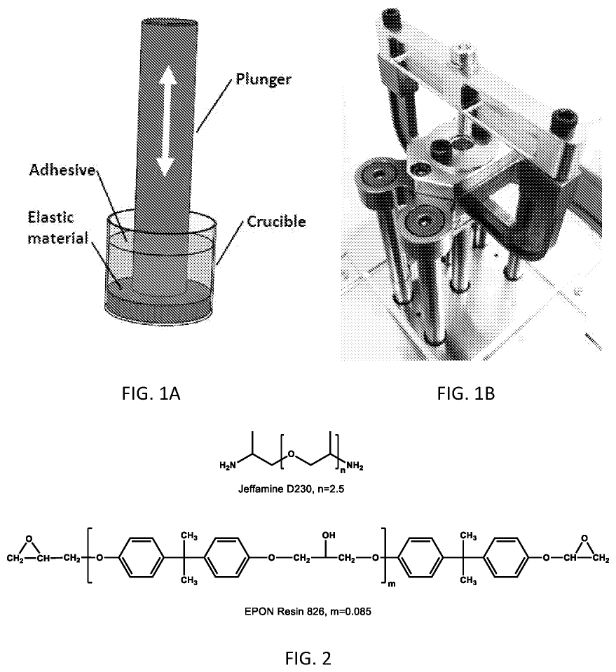

FIG. 1A presents a schematic of the C-KOM setup. The C-KOM is based on two main parts, a stationary crucible where a liquid (e.g., pre-cured) thermoset is placed, and a movable plunger which apply shear forces on the thermoset. The plunger is attached to a movable shaft of a dynamic mechanical analyzer (hereinbelow simply DMA) (commercially available from many sources). An elastic disc, made of room temperature vulcanizing (RTV) silicone, is placed at the bottom of the crucible. The plunger is preloaded against the elastic disc, applying oscillatory normal forces against it and shear forces against the thermoset. The elastic disc prevents the crucible from being deformed during the test. The forces applied by the DMA against the disc are of the order of the shear forces of the pre-cured thermoset and two orders of magnitude lower than the shear forces that are measured after the thermoset cures (after beginning of curing to solidification of the thermoset). These normal forces are subtracted from the total forces that are measured. FIG. 1B shows the C-KOM integrated with the DMA's bending fixture. A 100 μL crucible is attached using screws to the base of the fixture and the plunger, a 1.4 mm diameter stainless pin, is attached to the upper movable clamp. Before attaching the crucible to the stationary base, 50 μL of a well-mixed thermoset adhesive was inserted using a pipette. The crucible containing the adhesive was weighted by an analytical balance (precision of ±0.1 μg). By taking the adhesive density into consideration the exact volume of the adhesive was calculated for each measurement.

The material of choice to demonstrate the C-KOM ability to characterize the lifecycle of thermoset materials is a commercially available epoxy adhesive. Epoxy resins show excellent mechanical properties and high thermal and chemical resistance and they have a wide range of applications including coatings, electronics, electrical insulators, adhesives and fiber-reinforced composite materials.

The case study epoxy resin was EPON 826 DGEBA epoxy monomer (Momentive Inc.) and Jefamine D230 Poly (propylene glycol) bis (2-aminopropyl) ether (Huntsman Inc.) that was used as a crosslinker agent. FIG. 2 presents the molecular structure of the epoxy resin and of the Jeffamine D230 polyetheramine crosslinker. The resin and the crosslinker were mixed at a weight ratio of 3:1, respectively while maintaining a 1:1 molar ratio between the epoxide functional groups and the amine functional groups. High temperature PR-1006-60M RTV (Rotal Inc.) was used for the production of the elastic discs. The RTV was spread on a glass slide forming a 1 mm thick film and was cured for 24 h at room temperature (RT).

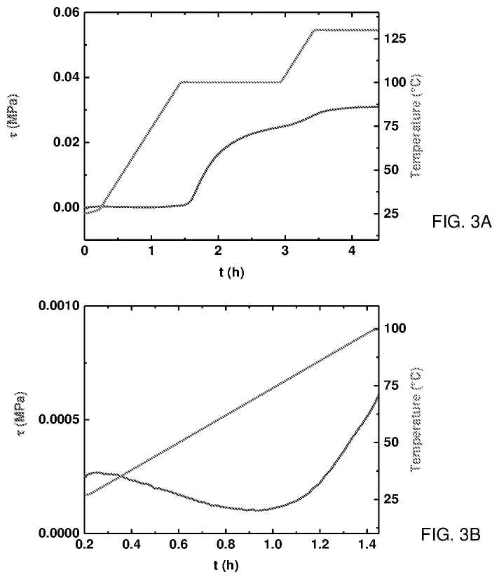

FIG. 3A presents a two-step curing process that was performed for the epoxy adhesive and was monitored by C-KOM. This process involves curing at 100° C. for 1.5 h and post curing at 130° C. for another 1 h. FIG. 3A presents the shear forces (t) that were measured by C-KOM during this process vs. the curing time (t). During the first 1.5 h from the beginning of the heating to 100° C. the changes in the shear forces are rather small. However, from that point on C-KOM measures a rapid increase in the shear forces, from 0.001 to 0.024 MPa, while the temperature is being held constant at 100° C. As the temperature was increased to 130° C., a further increase in the shear forces to 0.03 MPa is measured. After 1 h of post curing at 130° C. the trend of increase in the shear forces remains. This observation suggests that the curing process was not finished and may be governed by diffusion controlled rate.

FIG. 3B presents the C-KOM measurements during the first ˜1.5 h, as the freshly mixed adhesive was heated to 100° C. At the beginning of the heating stage C-KOM measures a decrease in the shear forces. This decrease is attributed to a decrease in the adhesive viscosity at higher temperatures. After ˜0.9 h the shear forces increase as the curing begins. As the adhesive start to cure the viscosity increases and the shear forces, measured by C-KOM, increase too. The results of FIG. 3 demonstrate the sensitivity of the C-KOM to small changes in viscosity. In addition it shows that in terms of the measured shear forces, the curing process requires longer curing periods or higher curing temperatures than previously thought. In order to address this issue a series of isothermal curing tests were performed using C-KOM.

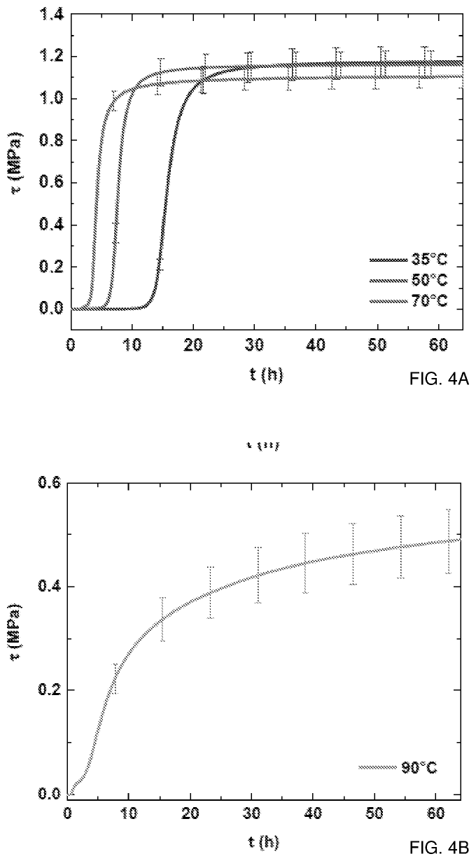

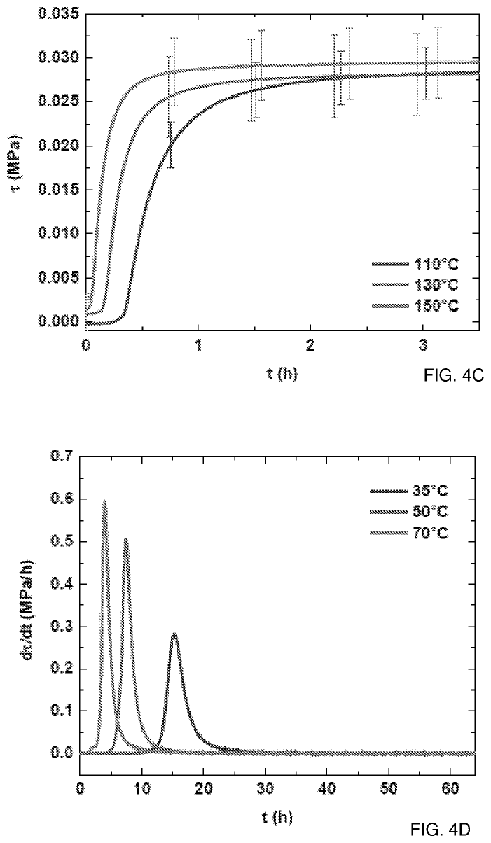

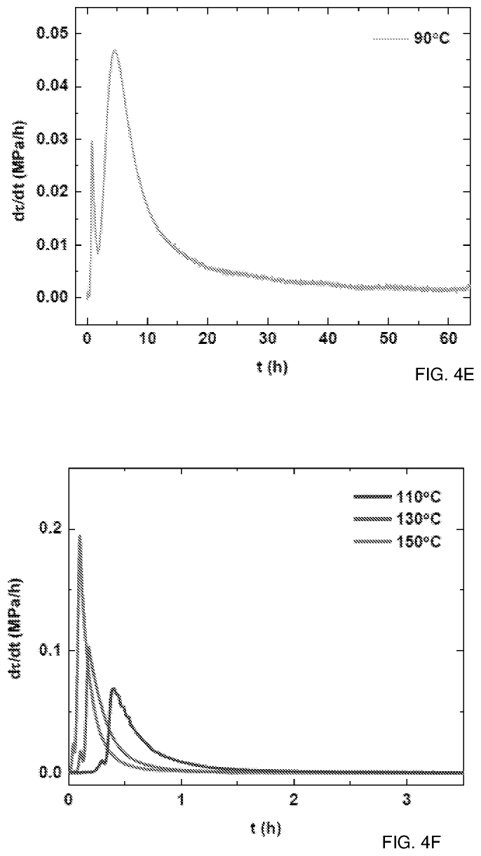

FIG. 4 presents in situ curing monitoring of τ (a-c) and the shear forces rate change over time (dτ/dt) (d-f) vs. t. at various isothermal curing temperatures, from 35 to 150° C. Each of these measurements was repeated three times. Regardless of the curing temperature the t vs. t measurements (FIG. 4A-C) present an s-shape profile. Such a profile is typical for curing of thermoset resins and rubbers and was found also in measurements performed by commercial rigid pendulum and torque rheometers. The first inflection in the t vs. t measurements is attributed to the gelation of the resin which corresponds to the irreversible transition from a liquid resin into an insoluble rubbery state adhesive. The second inflection is attributed to the vitrification which is defined as the stage at which the rate of curing reaction decreases intensely, and the reaction transform from a chemically controlled stage (rubbery state) to a diffusion controlled stage (glassy stage). As the curing temperature was increased the clear effect on the gelation and vitrification times is evident as the period to attain gelation and vitrification shortens from several hours time scale to minutes time scale. The final τ values also decrease dramatically by more than one order of magnitude as the curing temperature is increased. This is the result of the ratio between the curing temperature and the adhesive's T_{g}. If this ratio is larger than 1, that is if the curing temperature is higher than T_{g} the adhesive loses most of its mechanical properties as apparent by the τ values which decrease from ˜1 MPa at a temperature range of 35-70° C. to ˜0.03 MPa at a temperature range of 110-150° C.

The peaks in the dτ/dt vs. t graphs (FIG. 4D-F) represent the time needed for reaching the maximum curing rate for each of the iso-thermal curing temperatures. This time period was shortened as the curing temperature was increased. One can notice that from a curing temperature of 70° C. and above there are two peaks in the dτ/dt vs. t graphs; a small peak, as the curing begins, and a much larger one thereafter. At a curing temperature of 90° C. this phenomenon is more pronounced. The two peaks insinuate on two reaction rates that may take place as the adhesive starts to cure and solidify. It is thus suggested that at low curing temperatures of 35 and 50° C. the two reactions cannot be distinguished. At higher temperatures, from 70° C. and above, the two reactions can be noticed. At a curing temperature of 90° C. the rate of reactions is such that the two reactions can be clearly observed. The τ and dτ/t vs. t measurements are equivalent to the increase in the adhesive viscosity and the rate in which this viscosity increases. According to the Castro-Macosko viscosity equation the viscosity is a function of temperature, shear rate, and degree of cure. The cure characteristics of epoxy resin are a combination of an n^{th} order reaction and an autocatalytic reaction, having two separate reaction rates. At the early stage of the curing, the n^{th} order reaction is dominant. As the reaction proceeds, the reactants produce autocatalytic species that enhances the reaction rate. For amine-cured epoxy resins, it is accepted that two main addition reactions, described as primary and secondary amine ring-opening occur, and the two reaction rate constants are specific rate constants related to the primary and secondary amine-epoxy reactions. It is thus assumed that the two peaks shown on FIG. 4 d-f are associated with these two reactions.

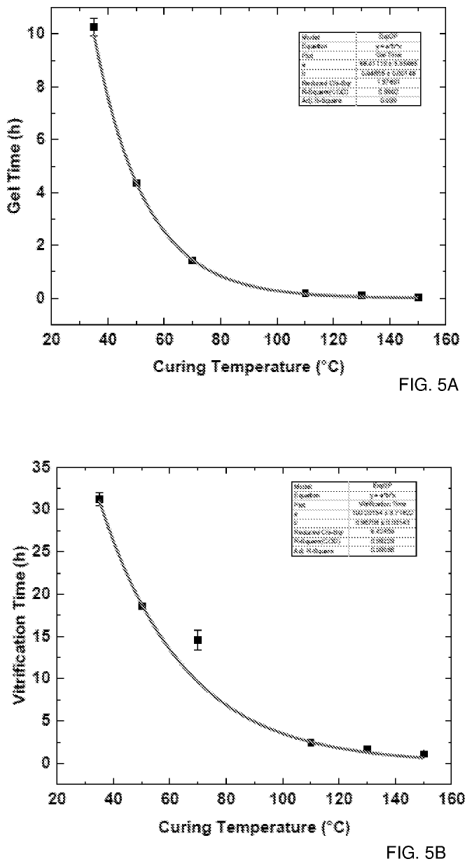

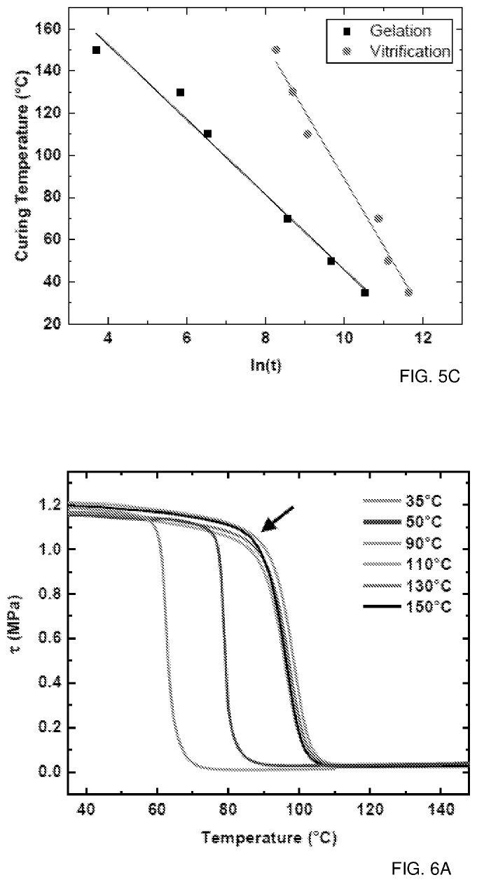

The gelation and vitrification times, taken from the inflection points of the τ vs. t iso-thermal curing curves (FIG. 4 a-c) are presented vs. the curing temperature, see FIGS. 5A and B, respectively. Both the gelation and the vitrification times decreased exponentially as the curing temperature was increased.

FIG. 5C presents a partial time-temperature-transformation (TTT) isothermal cure diagram that was built based on the data from FIGS. 5a and b. This TTT diagram, obtained by the linear motion of the C-KOM's plunger is comparable to TTT diagrams obtained by commercial parallel plate rheometer for epoxy resins.

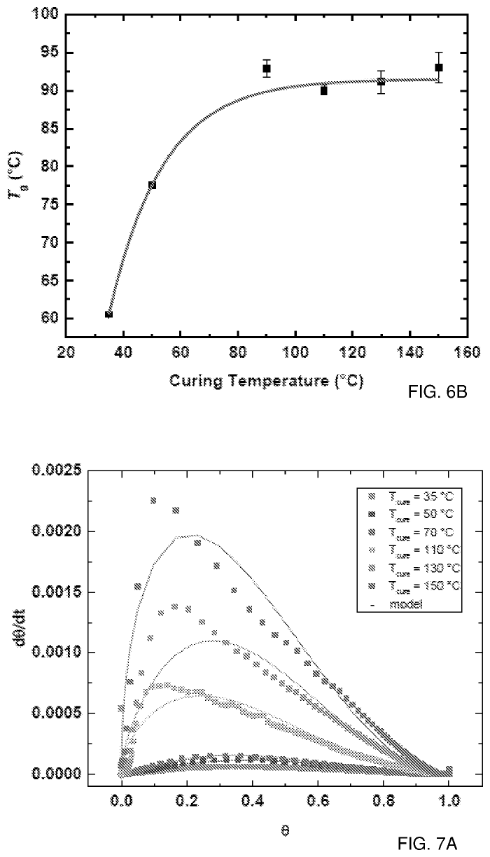

After each of the iso-thermal curing runs, presented in FIG. 4, the system was brought to RT and was slowly reheated while running the C-KOM and allowing the plunger to perform its linear oscillatory motion. FIG. 6 presents the r vs. temperature runs (a) and the T_{g} (onset) results vs. isothermal curing temperature (b), that were obtained from these runs. The results presented in FIG. 6a show a monotonical decrease in τ and a step function decrease at the onset of T_{g}, see arrow on FIG. 6A. This trend is typical to DMA storage modulus measurements of thermosets while their temperature is increased. The T_{g} values (FIG. 6B) show a logarithmic growth as the curing temperature was increased, up to a maximum T_{g} value (T_{g∞}). T_{g} increases at higher curing temperature until T_{g∞} is obtained. At this point the curing temperature has no effect on the T_{g}. The T_{g} increases as the crosslinking density of the thermoset resin increases. When the curing temperature exceeds T_{g∞} vitrification dramatically affects the progress of curing reaction, since the degree of conversion becomes practically quenched. The T_{g∞} obtained via C-KOM is 91° C.

The t vs t isothermal cure data, as presented in FIG. 4, were used to evaluate the curing kinetic parameters through a series of mathematical expressions and non-linear regressions. The degree of cure (θ), which is an indication of the epoxy resin crosslinking, was calculated from the time dependency of the t values, according to equation 1:

$$θ = \frac{{τ}_{t} - {τ}_{0}}{{τ}_{∞} - {τ}_{0}} \qquad ( 1 )$$

where τ₁ is the shear force measured by C-KOM at a given time, τ₀ is the shear force at the beginning of the measurement and τ_{∞} is the shear force at the end of the curing, where the shear forces become constant.

The curing reaction rate of thermoset materials can be described by two mechanisms, an n^{th} order reaction and an autocatalytic reaction in which the products of the cure reaction can catalyze the subsequent reaction between the resin and the hardener. In the n^{th} order reaction the curing reaction rate depends on the uncured portion of the reactants (1−θ), or on both the uncured and the cured portions of the reactants. The overall curing reaction rate can be considered as initially depend on the n^{th} order reaction followed by an autocatalytic reaction. The combination of the two reactions is given in equation 2. The first term corresponds to the n^{th} order reaction and the second term corresponds to the autocatalytic reaction.

$$\frac{{d}_{θ}}{{d}_{t}} = {{k}_{1} ( 1 - θ )}^{n} + {k}_{2} {{θ}^{m} ( 1 - θ )}^{n} \qquad ( )$$

where n and m are the reaction orders, k₁ and k₂ represent the temperature-dependent reaction rate constant, obeying the Arrhenius equation. The constants k₁ and k₂ may be specific rate constants related to the primary and secondary amine-epoxy reactions. The Arrhenius expression is given in equation 3:

$$k = A exp ( \frac{- {E}_{a}}{RT} ) \qquad ( 3 )$$

where A is a pre-exponential factor, Eₐ is the activation energy, R is the gas constant, and T is the reaction temperature. The initial stage of curing of an amine-based epoxy resin is temperature controlled. The final stage is diffusion controlled due to vitrification. The mobility of the functional groups becomes restricted at a certain degree of crosslinking and the curing reaction becomes diffusion controlled due to limited mobility of the reactants.

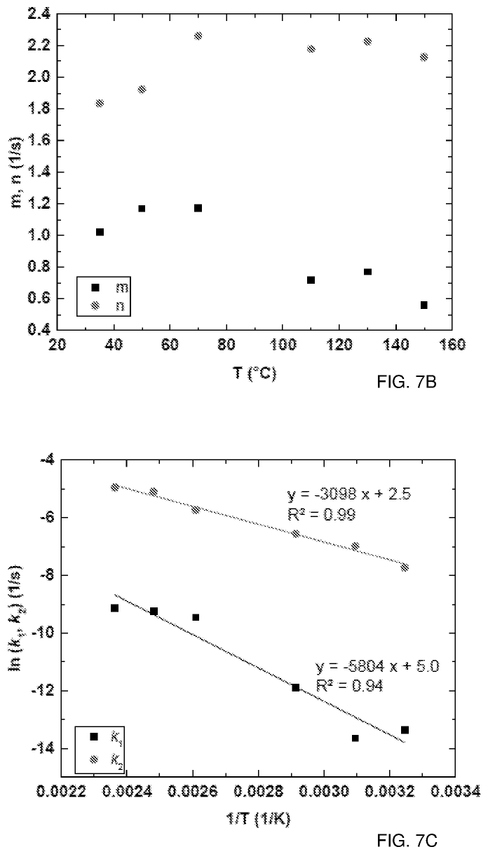

FIG. 7A presents a comparison between the experimental data and the model prediction of the curing reaction rate vs. the degree of cure at iso-thermal cure temperatures from 35 to 150° C. At an iso-thermal cure temperature of 70° C. and below the fitting between the experimental data and the model is excellent. However, at iso-thermal cure temperatures of 110° C. and above the model prediction of the dθ/dt values, especially as the dθ/dt value approaches its peak value, are less accurate. According to the experimental data obtained via C-COM (dθ/dₜ)ₘₐₓ values increased as the curing temperature was increased. It is also evident that as the curing temperature was increased (dθ/dt)ₘₐₓ values were received at lower 0 values. The reason for this is that as the curing temperature was increased, the curing rate increased too, and gelation and vitrification appeared earlier. As a result the diffusion limiting factor becomes dominant at an early stage of the curing reaction which shorten the period of obtaining (dθ/dt)ₘₐₓ values.

FIG. 7B presents Arrhenius plots of the reactions' order. As the curing temperature was increased m values decreased while n values increased. Such trends were reported in previous epoxy curing kinetics studies performed using ultrasonic and DSC methods. FIG. 7C presents Arrhenius plots of the reaction rate constants. Regardless of the curing temperature, in all cases the k₂ values are higher than the k₁ values. These results suggest that the autocatalytic reaction mechanism is the dominant curing mechanism rather than the non-autocatalytic mechanism. Following the calculation of the rate constants, the activation energies of the n^{th} order and the autocatalytic reactions were derived from the slopes of Arrhenius plots of the reaction rate constants. The activation energy on the non-autocatalytic curing mechanism (Eₐ₁) is 48.2 KJ/mol. The activation energy on the autocatalytic curing mechanism (Eₐ₂) is 25.8 KJ/mol.

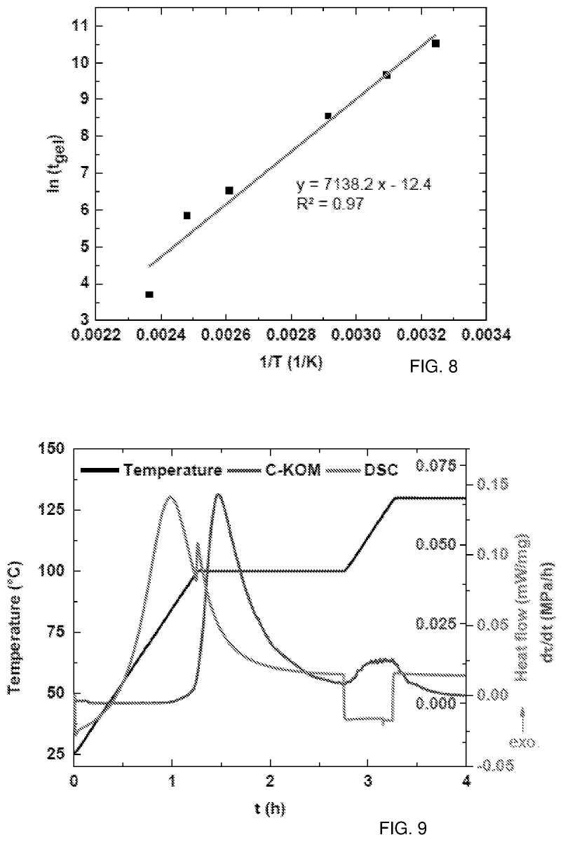

The degree of cure at gel time (t_{gel}) depends on the functionalities of the epoxy material system only. As such, it can be considered as a constant for a given epoxy system regardless of the cure temperature. The relationship between t_{gel} and the isothermal cure temperature (T) can be written as linear expression, as described in equation 4. The activation energy (Eₐ) can be calculated from the slope of ln(t_{gel}) vs. 1/T. FIG. 8 presents an Arrhenius type plot to obtain Eₐ of the crosslinking reaction, based on t_{gel} at different isothermal curing temperatures. A linear fit of the experimental data allows the calculation of Eₐ. The activation energy of the amine-based epoxy system in hand is 59.3 KJ/mol.

$$ln ( {t}_{g e l} ) = c + \frac{{E}_{a}}{R} \cdot \frac{1}{T} \qquad ( 4 )$$

The use of C-KOM is not limited to curing kinetics and iso-thermal curing studies. FIG. 9 presents a comparative thermo-analysis, between DSC and C-KOM, during curing and post-curing of the epoxy adhesive. In both cases freshly mixed adhesive was introduced into the systems and the curing process was performed. According to the DSC measurements the main exothermic peak appears after 1 h from the beginning of the curing stage, at a temperature of 85° C. At this stage of curing the C-KOM's dτ/dt values remain constant at their initial minimum values. However, 1.5 h from the beginning of curing C-KOM measured a peak in the dτ/dt values which indicates the gelation and vitrification of the adhesive, a process that started 30 min later than the heat flow peak measured by the DSC. During the post curing stage, as the adhesive was heated to 130° C., the exothermic peak from the DSC measurements can hardly be noticed. However, C-KOM shows a prominent peak in the dτ/dt values which indicates on substantial crosslinking during this stage. These results further exemplify the use of C-KOM as a tool for optimization of curing, especially during the post curing of thermosets, where DSC is less sensitive.

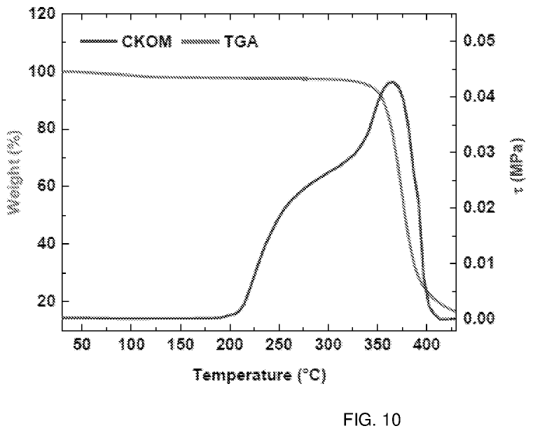

To further exemplify the versatility of C-KOM as a tool for investigation of the complete life cycle of a polymer, the inventors also investigated the thermal decomposition of the material and compared it with results obtained from conventional TGA system. FIG. 10 presents comparative thermal decomposition results, between TGA and C-KOM, during heating of the epoxy adhesive. As is the previous test in both cases freshly mixed adhesive was introduced into the systems and the curing temperature was gradually increased. According to the TGA results the onset of thermal decomposition is at a temperature of 355° C. According to C-KOM results gelation starts at a temperature of 185° C. As the temperature was further increased t values increase due to crosslinking of the adhesive as well as due to thermal expansion of the solidified adhesive. However, at a temperature of 362° C. a sharp decrease in r can be noticed due to thermal decomposition. The difference in the onset of thermal decomposition between TGA and C-KOM is only 7° C. These results demonstrate the versatility of C-KOM and its ability to be used for thermal decomposition measurements as well.

What is claimed is:

1. A system for dynamic mechanical analysis of a polymer, the system comprising:
a stationary crucible for receiving therein a polymer;
a movable plunger operative to apply shear forces on the polymer; and
a dynamic mechanical analyzer (DMA) comprising a movable shaft coupled to said plunger.

2. The system according to claim 1, further comprising an elastic disc placed at a bottom of said crucible, wherein said plunger is preloaded against said elastic disc and configured to apply oscillatory normal forces against said elastic disc and shear forces against said polymer.

3. The system according to claim 2, wherein forces applied by said DMA against elastic disc are of an order of the shear forces of said polymer and two orders of magnitude lower than shear forces that are measured after curing of said polymer.

4. The system according to claim 1, wherein said plunger is movable in oscillatory axial motion.

5. The system according to claim 1, wherein said plunger is configured to apply the shear forces on the polymer while the polymer is in a liquid state.

6. The system according to claim 1, wherein said plunger is configured to apply the shear forces on the polymer while the polymer is in a solid state.

7. The system according to claim 1, wherein said plunger is configured to apply the shear forces on the polymer while the polymer is in a transition between a liquid state and a solid state.

8. A method for dynamic mechanical analysis of a polymer, the method comprising:
placing a polymer in a stationary crucible;
applying shear forces on the polymer with a movable plunger; and
performing dynamic mechanical analysis of said polymer with a dynamic mechanical analyzer comprising a movable shaft coupled to said plunger.

9. The method according to claim 8, wherein an elastic disc is placed at a bottom of said crucible, and comprising preloading said plunger against said elastic disc and applying oscillatory normal forces against said elastic disc and shear forces against said polymer.

10. The method according to claim 9, wherein forces applied by said DMA against elastic disc are of an order of the shear forces of said polymer and two orders of magnitude lower than shear forces that are measured after curing of said polymer.

11. The method according to claim 8, wherein said plunger applies the shear forces on the polymer while the polymer is in a liquid state.

12. The method according to claim 8, wherein said plunger applies the shear forces on the polymer while the polymer is in a solid state.

13. The method according to claim 8, wherein said plunger applies the shear forces on the polymer while the polymer is in a transition between a liquid state and a solid state.

14. The method according to claim 8, further comprising exposing said crucible to variable temperatures.

15. The method according to claim 8, further comprising irradiating said crucible with electromagnetic radiation.
